(19) United States
(12) Patent Application Publication
(10) Pub. No.: US 2009/0012399 A1
(43) Pub. Date: Jan. 8, 2009

(54) ULTRASONIC DIAGNOSTIC APPARATUS

(76) Inventors: Kazuhiro Sunagawa, Miyagi (JP); Yoshinao Tannaka, Kanagawa (JP); Makoto Kato, Kanagawa (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(21) Appl. No.: 11/815,581

(22) PCT Filed: Feb. 6, 2006
(86) PCT No.: PCT/JP2006/301995

§ 371 Date: Aug. 6, 2007

(30) Foreign Application Priority Data
Feb. 7, 2005 (JP) 2005-030533

(51) Int. Cl. A61B 8/00

(52) U.S. Cl. 600/454

(57) ABSTRACT

The ultrasonic diagnostic apparatus of the invention evaluates a shape or qualitative property of an organism's arterial wall tissue and includes: a delay control section 3 for controlling delays for ultrasonic vibrators 1 in an ultrasonic probe 2; a transmitting section 5 for driving the probe under the control of the control section 3 such that the probe 2 transmits a first ultrasonic beam toward different locations within a scan region, defined along the axis of the artery, every predetermined frame period; a receiving section 6 for receiving ultrasonic echoes, generated by getting the first beam reflected by the wall, at the probe every set of frame periods, thereby outputting a first group of ultrasonic echo signals; and a signal processing section 13 for calculating a thickness variation, or elasticity, of the tissue between measuring points on the tissue in response to the first group of echo signals. The section 13 selects one of the echo signals of the first group every frame period according to an axial velocity of the tissue to make calculations at each measuring point.

### TECHNICAL FIELD

The present invention relates to an ultrasonic diagnostic apparatus and more particularly relates to an ultrasonic diagnostic apparatus for calculating a variation in the thickness or the elasticity of an arterial wall.

### BACKGROUND ART

A Doppler technique for detecting a frequency deviation by taking advantage of the Doppler effect of an ultrasonic echo signal is known as a method for measuring the motion velocity or the magnitude of displacement of a living tissue with ultrasonic waves. For example, Patent Document No. 1 discloses a method for measuring the blood flow rate by the Doppler technique. Also, to accurately analyze the frequency of an ultrasonic echo signal with a frequency deviation, Non-Patent Document No. 1 proposes adopting a fast Fourier transform (FFT), while Patent Documents Nos. 2 and 3 propose adopting an autocorrelation method.

The measurements can be done relatively easily according to the Doppler technique. However, no Doppler effect should be produced in an ultrasonic echo that has been reflected perpendicularly to the direction in which the living tissue is moving, which is a problem. In other words, the motion velocity of the living tissue cannot be detected by the Doppler technique perpendicularly to the ultrasonic echo. To overcome such a problem, Patent Documents Nos. 4 through 7 disclose methods for detecting a complete two- or three-dimensional motion of a living tissue using multiple ultrasonic beams with mutually different angles of deviation.

Meanwhile, Patent Document No. 8 discloses a method for accurately estimating the kinetic momentum of a measuring point by calculating the phase shift of an ultrasonic echo signal precisely by a minimum square method. According to this method, the variation in the thickness (i.e., the magnitude of strain) of a living tissue can be figured out based on the kinetic momentum of each portion of the living tissue. The living tissue consists of elastic fibers, collagen fibers, fat, clot and so on, which have respectively different elasticities. That is why by calculating the elasticity based on the thickness variation caused when a stress is applied to an organism's internal tissue, the constitution of the tissue can be identified and the status of the diseased tissue can be estimated based on the elasticity value.

Recently, the number of people suffering from various cardiovascular diseases, including heart infarction and brain infarction, has been on the rise, thus making it more and more urgent to prevent and treat these diseases. The onset of heart or brain infarction is closely correlated to atherosclerosis. For that reason, if the elasticity of an arterial wall tissue can be measured with an ultrasonic diagnostic apparatus as described above, the degree of advancement of atherosclerosis can be determined early, which would contribute to preventing or treating these diseases. That is why development of ultrasonic diagnostic apparatuses that can measure the elasticity of an arterial wall tissue is awaited.
Patent Document No. 1: Japanese Patent Application Laid-Open Publication No. 2001-070305
Patent Document No. 2: Japanese Patent Gazette for Opposition No. 62-44494
Patent Document No. 3: Japanese Patent Application Laid-Open Publication No. 6-114059
Patent Document No. 4: Japanese Patent Application Laid-Open Publication No. 5-115479
Patent Document No. 5: Japanese Patent Application Laid-Open Publication No. 10-262970
Patent Document No. 6: U.S. Pat. No. 6,770,034
Patent Document No. 7: U.S. Pat. No. 6,258,031
Patent Document No. 8: Japanese Patent Application Laid-Open Publication No. 10-5226
Non-Patent Document No. 1: “Revised Version of Medical Ultrasonic Equipment Handbook”, edited by Electronic Industries Association of Japan, Corona Publishing Co., Ltd., Jan. 20, 1997, pp. 116-123

### DISCLOSURE OF INVENTION

### Problems to be Solved by the Invention

The artery dilates and contracts radially responsive to a variation in the flow rate or the pressure of the blood flowing there. That is why by making an ultrasonic beam enter the artery perpendicularly to its axis and receiving an ultrasonic echo, the variation in the thickness of the arterial wall tissue could be measured and the elasticity thereof could be calculated on a cross section that includes the axis of the artery.

However, the present inventors discovered as a result of exhaustive experiments that the arterial wall sometimes slightly moved axially in sync with the termination of one cardiac cycle. We also discovered that the arterial wall did not always show an observable axial motion and sometimes showed almost no axial motion depending on the specific measuring point or the condition of the person under test.

Anyway, if the arterial wall is moving axially, the elasticity that has been calculated on the supposition that the wall is not moving axially is not accurate but should contain some error. However, as long as it is uncertain if the arterial wall is actually moving axially, it is difficult to determine whether the elasticity calculated is accurate or not.

If the arterial wall is actually moving axially, the elasticity could be calculated more accurately by precisely measuring the two-dimensional motion of the arterial wall on a cross section that contains the axis of the artery. The elasticity could be calculated by precisely analyzing the motion of the arterial wall by one of the methods disclosed in Patent Documents Nos. 4 through 7, for example. To measure the two-dimensional motion by any of these methods, however, a large scale measuring circuit would be needed and a huge amount of computations should be done in order to track the target measuring point. Among other things, the computations to be done to calculate the thickness variation or elasticity of a living tissue is far more complex than the computations to be done to calculate the motion velocity of a measuring point. That is why it is very difficult for a normal computer included in a conventional ultrasonic diagnostic apparatus to get that huge amount of computations done. Also, an ultrasonic diagnostic apparatus including a computer with very high computational performance would be outrageously expensive.

In order to overcome the problems described above, the present invention has an object of providing an ultrasonic diagnostic apparatus that can accurately measure the thickness variation or elasticity of a living tissue with a simple computing circuit in view of the axial motion of the arterial wall.

### Means for Solving the Problems

An ultrasonic diagnostic apparatus according to the present invention evaluates a shape property or a qualitative property of an arterial wall tissue of an organism. The apparatus includes: a delay control section for controlling delays for respective ultrasonic vibrators included in an ultrasonic probe; a transmitting section for driving the ultrasonic probe under the control of the delay control section such that the ultrasonic probe transmits a first ultrasonic beam toward multiple different locations within a scan region, which is defined along the axis of the organism's artery, every predetermined frame period; a receiving section for receiving a plurality of ultrasonic echoes, generated by getting the first ultrasonic beam reflected by the arterial wall, at the ultrasonic probe every predetermined frame period, thereby outputting a first group of ultrasonic echo signals; and a signal processing section for calculating a thickness variation, or the elasticity, of the arterial wall tissue between multiple measuring points that have been set on the arterial wall tissue in response to the first group of ultrasonic echo signals. The signal processing section selects one of the ultrasonic echo signals of the first group every frame period according to an axial velocity of the arterial wall tissue to make calculations at each said measuring point.

In one preferred embodiment, the signal processing section includes a motion velocity detecting section, the transmitting section transmits a second ultrasonic beam, the receiving section outputs a second group of ultrasonic echo signals, which are generated by getting the second ultrasonic beam reflected by the arterial wall, and the motion velocity detecting section calculates the axial velocity of the arterial wall tissue based on the second group of ultrasonic echo signals.

In this particular preferred embodiment, the first and second ultrasonic beams have mutually different angles of deviation.

In a specific preferred embodiment, the delay control section changes, at regular intervals, delay control rates to transmit the second ultrasonic beam.

In another preferred embodiment, the delay control section receives a biomedical signal, containing information about the organism, and changes delay control rates to transmit the second ultrasonic beam at an interval that agrees with one cycle of the biomedical signal.

In a specific preferred embodiment, the cycle of the biomedical signal is a cardiac cycle.

In another preferred embodiment, the first ultrasonic beam is substantially perpendicular to the axis of the artery and the second ultrasonic beam is not perpendicular to the axis of the artery.

In still another preferred embodiment, the measuring points are arranged two-dimensionally, and the computing section calculates the thickness variation, or the elasticity, of the arterial wall tissue two-dimensionally.

In this particular preferred embodiment, the ultrasonic diagnostic apparatus further includes a display section for presenting results of calculations done by the computing section as a two-dimensional map.

An ultrasonic diagnostic apparatus controlling method according to the present invention is a method for getting an ultrasonic diagnostic apparatus controlled by a control section of the apparatus. The method includes the steps of: transmitting a first ultrasonic beam from an ultrasonic probe toward multiple different locations within a scan region, which is defined along the axis of an organism's artery, every predetermined frame period; receiving a plurality of ultrasonic echoes, generated by getting the first ultrasonic beam reflected by the arterial wall of the artery, at the ultrasonic probe every predetermined frame period, thereby generating a first group of ultrasonic echo signals; selecting one of the ultrasonic echo signals of the first group every frame period according to an axial velocity of the arterial wall tissue to make calculations at each measuring point; and calculating a thickness variation, or the elasticity, of the arterial wall tissue between at least two of multiple measuring points that have been set on the arterial wall tissue in response to the ultrasonic echo signal selected from the first group.

In one preferred embodiment, the step of calculating includes the steps of: transmitting a second ultrasonic beam toward the artery to obtain a second group of ultrasonic echo signals, which are generated by getting the second ultrasonic beam reflected by the arterial wall, and calculating the axial velocity of the arterial wall tissue based on the second group of ultrasonic echo signals.

In this particular preferred embodiment, the method includes the step of transmitting the second ultrasonic beam at an interval that agrees with one cycle of a biomedical signal containing information about the organism.

In a specific preferred embodiment, the cycle of the biomedical signal is a cardiac cycle.

Another ultrasonic diagnostic apparatus according to the present invention evaluates a shape property or a qualitative property of an arterial wall tissue of an organism. The apparatus includes: a delay control section for controlling delays for respective ultrasonic vibrators included in an ultrasonic probe; a transmitting section for driving the ultrasonic probe under the control of the delay control section such that the ultrasonic probe transmits not only a first ultrasonic beam toward multiple different locations within a scan region, which is defined along the axis of the organism's artery, but also a second ultrasonic beam toward the organism's artery at a different angle of deviation from the first ultrasonic beam; a receiving section for receiving a plurality of ultrasonic echoes, generated by getting the first ultrasonic beam reflected by the arterial wall, as well as the second ultrasonic beam, at the ultrasonic probe, thereby outputting first and second groups of ultrasonic echo signals; a signal processing section for calculating a thickness variation, or the elasticity, of the arterial wall tissue between multiple measuring points that have been set on the arterial wall tissue in response to the first group of ultrasonic echo signals and for detecting either the axial velocity or the magnitude of axial displacement of the arterial wall between the measuring points in response to the second group of ultrasonic echo signals; and a display section for presenting either the thickness variation or the elasticity thereon. The display section changes the modes of presenting the thickness variation or the elasticity on itself according to the motion velocity or the magnitude of displacement of the arterial wall tissue.

In one preferred embodiment, if the axial velocity or the magnitude of axial displacement of the arterial wall tissue is equal to or greater than a predetermined threshold value, the signal processing section outputs neither the thickness variation nor the elasticity of its associated tissue to the display section.

In an alternative preferred embodiment, if the axial velocity or the magnitude of axial displacement of the arterial wall tissue is equal to or greater than a predetermined threshold value, the signal processing section sets either the thickness variation or the elasticity of its associated tissue to a predetermined value and outputs the value to the display section.

In another alternative preferred embodiment, if the axial velocity or the magnitude of axial displacement of the arterial wall tissue is equal to or greater than a predetermined threshold value, the signal processing section gets predetermined character or graphic information presented on the display section.

In still another alternative preferred embodiment, if the axial velocity or the magnitude of axial displacement of the arterial wall tissue is equal to or greater than a predetermined threshold value, the signal processing section calculates the average of thickness variations or elasticities of multiple arterial wall tissues along their axis and outputs the average to the display section.

In yet another alternative preferred embodiment, if the axial velocity or the magnitude of axial displacement of the arterial wall tissue is equal to or greater than a predetermined threshold value, the signal processing section determines the number of arterial wall tissues, for which the average of their thickness variations or elasticities should be worked out along their axis, by the motion velocity or the magnitude of displacement, gets the average of the thickness variations or elasticities of the determined number of tissues and then outputs the average to the display section.

### EFFECTS OF THE INVENTION

According to the present invention, an ultrasonic echo signal for calculating a thickness variation or elasticity between measuring points based on the axial velocity of an arterial wall tissue is selected every frame period. The motion velocity at each measuring point may be calculated as in the conventional method using the selected ultrasonic beam. As a result, the shape property or the qualitative property of an organism's arterial tissue can be evaluated accurately without significantly increasing the computational complexity. That is why there is no need to use a computing circuit with high computing performance for an ultrasonic diagnostic apparatus. Consequently, an ultrasonic diagnostic apparatus that can measure the elasticity accurately can be provided at a reduced cost.

### BRIEF DESCRIPTION OF DRAWINGS

FIG. 1 is a block diagram showing a first preferred embodiment of an ultrasonic diagnostic apparatus according to the present invention.

FIG. 2 schematically shows ultrasonic beams transmitted from an ultrasonic probe.

FIG. 3 is a flowchart showing a procedure of making measurements with the ultrasonic diagnostic apparatus shown in FIG. 1.

FIG. 4 is a schematic representation illustrating an axial motion of an arterial wall tissue in an organism.

FIG. 5 is a schematic representation showing how to select an ultrasonic beam on a frame-by-frame basis.

FIG. 6 is a schematic representation illustrating how to calculate the axial velocity of an arterial wall tissue using a second ultrasonic beam.

FIGS. 7(a) and 7(b) are schematic representations showing the timings to transmit first and second ultrasonic beams.

FIG. 8 shows measuring points on an ultrasonic beam.

FIG. 9 shows how to calculate the magnitudes of dilation or contraction between the measuring points.

Portions (a) through (f) of FIG. 10 show a waveform of the vibration velocity of an arterial wall in one cardiac cycle, an electrocardiographic complex, a waveform of a blood flow velocity, a variation in the inside diameter of a blood vessel, a variation in the thickness of a vascular wall and an axial displacement.

FIGS. 11(a) and 11(b) show the locations of tissues under test at a time t₁ when the vascular wall has the greatest thickness and at a time t₂ when the vascular wall has the smallest thickness.

FIG. 12 is a block diagram showing a second preferred embodiment of an ultrasonic diagnostic apparatus according to the present invention.

FIG. 13 is a flowchart showing a procedure of making measurements with the ultrasonic diagnostic apparatus shown in FIG. 12.

FIG. 14 schematically illustrates exemplary images presented on the display section of the ultrasonic diagnostic apparatus shown in FIG. 12.

FIG. 15 schematically illustrates other exemplary images presented on the display section of the ultrasonic diagnostic apparatus shown in FIG. 12.

FIG. 16 is a flowchart showing another procedure of making measurements with the ultrasonic diagnostic apparatus shown in FIG. 12.

FIG. 17 schematically illustrates exemplary images presented on the display section of an ultrasonic diagnostic apparatus that operates following the procedure shown in FIG. 16.

FIG. 18 schematically illustrates other exemplary images presented on the display section of the ultrasonic diagnostic apparatus that operates following the procedure shown in FIG. 16.

### DESCRIPTION OF REFERENCE NUMERALS

1 ultrasonic vibrator
3 ultrasonic probe
3 delay control section
4 delay control rate storage section transmitting section
6 receiving section
7 received signal storage section
8 motion velocity detecting section
9 computing section
10 display section
11 control section
12 storage section
13 signal processing section
14 image generating section
20 ultrasonic diagnostic apparatus
31 biomedical signal detecting section
51 tomographic image
61 artery anterior wall
62 blood vessel lumen
63 artery posterior wall
A, A1, . . . An first ultrasonic beam
B second ultrasonic beam

### BEST MODE FOR CARRYING OUT THE INVENTION

### Embodiment 1

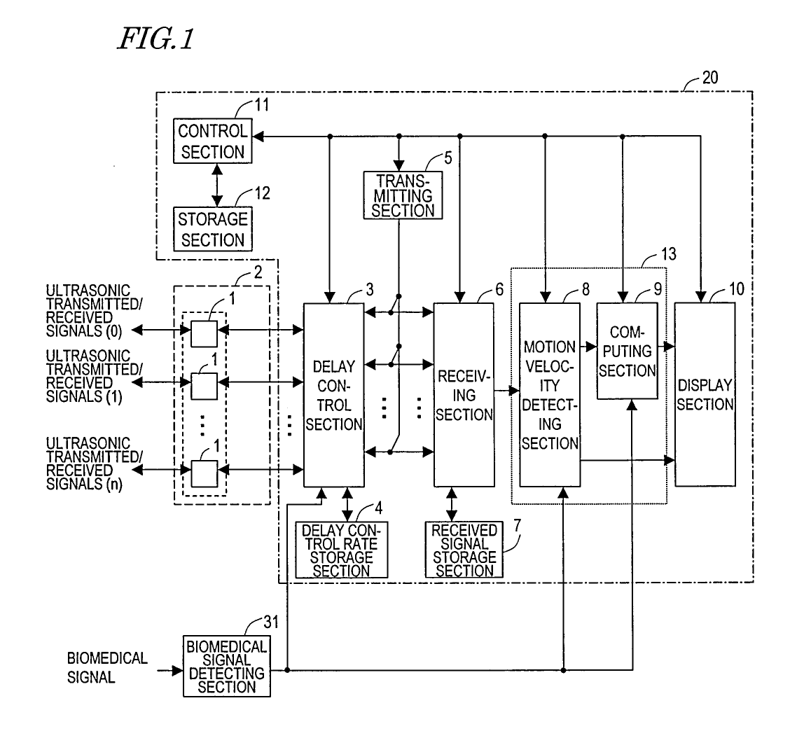

Hereinafter, a First Preferred Embodiment of an ultrasonic diagnostic apparatus according to the present invention will be described with reference to the accompanying drawings. FIG. 1 is a block diagram of an ultrasonic diagnostic apparatus 20. The ultrasonic diagnostic apparatus 20 evaluates either a shape property or a qualitative property of an organism using an ultrasonic probe 2. The apparatus 20 can be used particularly effectively to measure the elasticity of an arterial wall tissue of an organism, among other things. As used herein, a “shape property” of an organism may refer to either the shape of a living tissue or the motion velocity of the living tissue due to a variation in its shape with time, the magnitude of its displacement, which is an integrated value thereof, and a variation in thickness between two points that have been set on the living tissue. On the other hand, a “qualitative property” of an organism will refer herein to the elasticity of the living tissue, for example. The ultrasonic diagnostic apparatus 20 includes a delay control section 3, a delay control rate storage section 4, a transmitting section 5, a receiving section 6, a received signal storage section 7, a signal processing section 13, a display section 10, a control section 11, and a storage section 12.

The ultrasonic probe 2 includes a plurality of ultrasonic vibrators 1 and is used to transmit an ultrasonic beam toward an arterial wall tissue, which is the object of measurement, and to receive an ultrasonic echo, which is generated by getting the transmitted ultrasonic beam reflected by the arterial wall tissue. As will be described in detail later, the ultrasonic probe 2 preferably includes a plurality of ultrasonic vibrators 1, which are arranged at least one-dimensionally. The ultrasonic probe 2 is connected to the delay control section 3.

The transmitting section 5 drives the respective ultrasonic vibrators 1 of the ultrasonic probe 2, thereby generating an ultrasonic transmission signal to transmit an ultrasonic beam to the arterial wall tissue. The ultrasonic transmission signal thus generated is input to the delay control section 3, where the delays are controlled such that the respective ultrasonic vibrators 1 are driven at predetermined timings. In this manner, an ultrasonic beam is transmitted to the arterial wall tissue. The ultrasonic transmission signals generated by the transmitting section 5 include a signal to evaluate a shape property or a qualitative property of the arterial wall tissue, which is the object of measurement, and a signal to figure out the axial velocity (velocity in an axial direction) of the arterial wall tissue.

That is why the ultrasonic beams transmitted from the ultrasonic probe 2 also include a beam to evaluate a shape property or a qualitative property of the arterial wall tissue and a beam to figure out the axial velocity of the arterial wall tissue. These beams will be referred to herein as a “first ultrasonic beam” and a “second ultrasonic beam”, respectively.

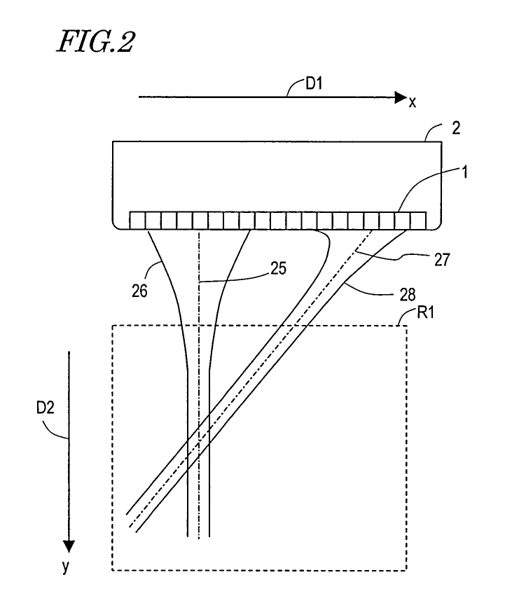

FIG. 2 schematically shows ultrasonic beams transmitted from the ultrasonic probe 2. When the ultrasonic transmitted signal generated by the transmitting section 5 is subjected to the delay control by the delay control section 3, a number of (e.g., ten plus to several tens of) ultrasonic vibrators 1 included in the ultrasonic probe 2 generate a single ultrasonic beam 26 with an acoustic line 25. The ultrasonic vibrators 1 are arranged one-dimensionally. That is why by sequentially shifting that combination of the ultrasonic vibrators 1 to drive in their arrangement direction as pointed by the arrow D1, the location of the first ultrasonic beam 26 can be sequentially shifted in the direction in which the ultrasonic vibrators 1 are arranged. As a result, the tissue can be scanned with the first ultrasonic beam 26, and the shape or qualitative property of the arterial wall tissue can be evaluated in a two-dimensional scan region R1, which is defined by the scan direction (as pointed by the arrow D1) and the depth direction (as pointed by the arrow D2) of the first ultrasonic beam 26. The scan region R1 will be referred to herein as a “frame” and one period in which the first ultrasonic beam 26 completes its scan will be referred to herein as a “frame period”. To evaluate the shape or qualitative property of the arterial wall tissue, a number of scan regions R1 are preferably scanned per second with the first ultrasonic beam 26.

As shown in FIG. 2, a second ultrasonic beam 28 with an acoustic line 27 is transmitted at a different angle of deviation from the first ultrasonic beam 26. The first ultrasonic beam 26 is preferably transmitted from the ultrasonic probe 2 such that its acoustic line 25 is perpendicular to the axis of the arterial wall tissue. On the other hand, the second ultrasonic beam 28 is preferably transmitted from the ultrasonic probe 2 such that its acoustic line 27 is not perpendicular to the axis of the arterial wall tissue.

The ultrasonic echoes, reflected from the arterial wall toward the ultrasonic probe 2, are received at the respective ultrasonic vibrators 1 of the ultrasonic probe 2, have their delays controlled by the delay control section 3, and then are synthesized and amplified by the receiving section 6, which outputs the synthesized ultrasonic echo signal to the signal processing section 13. The signal obtained by synthesizing the ultrasonic echoes, generated by getting the first ultrasonic beam reflected, and the signal obtained by synthesizing the ultrasonic echoes, generated by getting the second ultrasonic beam reflected, will be referred to herein as a “first ultrasonic echo signal” and a “second ultrasonic echo signal”, respectively.

Every time an ultrasonic beam is transmitted from the ultrasonic probe 2, the delay control section 3 performs a delay control by reference to the delay control rates of the respective ultrasonic vibrators 1, which are stored in advance in the delay control rate storage section 4, in transmitting an ultrasonic wave and in receiving an ultrasonic echo. Meanwhile, the ultrasonic echo signal, synthesized by the receiving section 6, is stored in the received signal storage section 7, which preferably has a storage capacity that is ample enough to store the first (type of) ultrasonic echo signals for multiple frames.

The signal processing section 13 includes a motion velocity detecting section 8 and a computing section 9. The motion velocity detecting section 8 detects the motion velocity of the arterial wall tissue at each measuring point, or the magnitude of displacement that is an integrated value thereof, based on the first ultrasonic echo signal. Also, the motion velocity detecting section 8 detects the axial velocity of the arterial wall tissue, or the magnitude of axial displacement thereof, based on the second ultrasonic echo signal.

Based on the motion velocities, or the magnitudes of displacement, at respective measuring points on the arterial wall tissue, which have been derived from the first ultrasonic echo signals, the computing section 9 calculates either the thickness variation, or the elasticity, between the measuring points on the arterial wall tissue. In this case, based on the axial velocity, or magnitude of axial displacement, of the arterial wall tissue that has been derived from the second ultrasonic echo signal, the computing section 9 selects a first ultrasonic echo signal to calculate the thickness variation or the elasticity for each arterial wall tissue between the measuring points every frame period. By performing calculations using the first ultrasonic echo signal that has been selected in this manner, the thickness variation or the elasticity of the arterial wall tissue can be calculated in view of the axial motion of the arterial wall tissue.

The motion velocity detecting section 8 may detect the motion velocity at each measuring point by any of normally used methods including FFT Doppler technique and autocorrelation technique. However, by adopting a restricted minimum square method as will be described in detail later, the thickness variation or elasticity of an even smaller region can also be calculated.

The display section 10 presents the shape property such as the motion velocity or the thickness variation and/or the qualitative property such as the elasticity, which have been calculated by the signal processing section 13 for each portion of the arterial wall tissue. Depending on the measuring point, these values may be presented as a two-dimensional map or superimposed on a B-mode tomographic image, which is one of basic functions that an ordinary ultrasonic diagnostic apparatus has. Optionally, the shape property and qualitative property may be presented in gray scales or color tones associated with the property figured out.

The control section 11 controls the overall ultrasonic diagnostic apparatus 20. Specifically, the control section 11 controls the delay control section 3, transmitting section 5, receiving section 6, signal processing section 13 and display section 10, and stores information and control information, provided by the delay control section 3, transmitting section 5, receiving section 6, signal processing section 13 and display section 10, in the storage section 12.

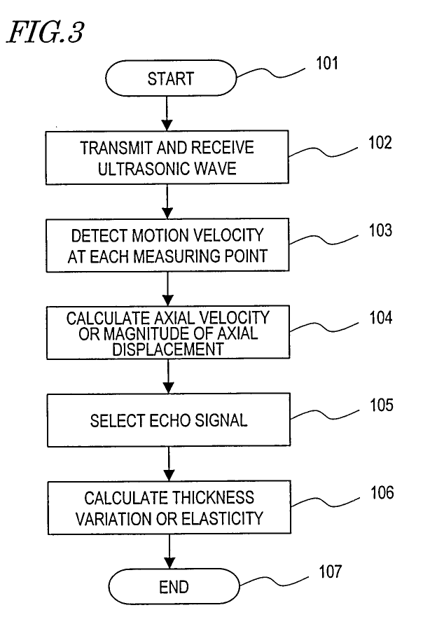

Specifically, the ultrasonic diagnostic apparatus 20 makes measurements following the procedure of the flowchart shown in FIG. 3. First, in Step 102, the transmitting section 5 transmits an ultrasonic wave from the ultrasonic probe 2 toward an organism including an arterial blood vessel. The ultrasonic echoes, generated by getting the transmitted ultrasonic wave reflected by the organism, are received through the ultrasonic probe 2 at the receiving section 6. The ultrasonic wave transmitted includes first and second ultrasonic beams. And the receiving section 6 outputs first and second ultrasonic echo signals based on these reflected echoes.

Next, in Step 103, the motion velocity detecting section 8 of the signal processing section 13 detects either the motion velocity or the magnitude of displacement at each measuring point on the arterial wall tissue based on the first ultrasonic echo signal. The motion velocity or the magnitude of displacement obtained in this processing step includes only components that are parallel to the ultrasonic beam. That is why the motion velocity or the magnitude of displacement at each of multiple measuring points, which has been calculated based on one of multiple ultrasonic echo signals of the first group generated as a result of the scan by the first ultrasonic beam 26, can be independent of those calculated based on the other ultrasonic echo signals of the first group.

Subsequently, in Step 104, the motion velocity detecting section 8 detects the axial velocity of the arterial wall based on the second ultrasonic echo signal and may also calculate the magnitude of axial displacement. Thereafter, in Step 105, the computing section 9 selects one of the ultrasonic echo signals of the first group for calculating the thickness variation or elasticity between the measuring points on the arterial wall tissue according to the axial velocity or the magnitude of axial displacement of the arterial wall as will be described in detail later. Then, in Step 106, based on the motion velocity or magnitude of displacement that has been calculated for each measuring point on the ultrasonic echo signal selected from the first group, the computing section 9 calculates the thickness variation or elasticity between the measuring points on the arterial wall tissue.

The step 102 of transmitting and receiving an ultrasonic wave is repeatedly performed a number of times during the measurement. The processing steps 102 through 106 are performed multiple times, too. It should be noted that the processing steps 103 and 104 do not have to be carried out in this order but may be performed in parallel or in reverse order.

Hereinafter, the principle of measurements done by the ultrasonic diagnostic apparatus 20 will be described in detail. FIG. 4 schematically illustrates an artery 31 in a living tissue 30. As shown in FIG. 4, when the heart contracts, blood pumps out from the heart periodically, thus producing a blood flow F. The blood flowing through the artery 31 is subjected to a pressure P. Due to the pressure P, the artery 31 contracts and dilates periodically. And as the artery 31 dilates, its vascular wall decreases its thickness. This is a motion produced in y direction, which is perpendicular to the axial direction of the artery 31 as shown in FIG. 4. Meanwhile, the blood flow F produces a shear stress Q on the vascular wall of the artery 31. As a result, the vascular wall of the artery 31 is also displaced along the axis of the artery 31 due to the shear stress Q. If the measuring region on the artery 31 is located close to the heart, the artery 31 may also be physically displaced axially as the heart contracts. These motions are produced along the axis of the artery 31 (i.e., in x direction). And these motions of the artery 31 in the axial direction and in the direction perpendicular to the axial direction are repeated at an interval that agrees with one cardiac cycle.

As shown in FIG. 4, if the shape property or qualitative property of the artery 31 is evaluated, the ultrasonic probe 2 is arranged with respect to the artery 31 such that the arrangement direction of the ultrasonic vibrators 1 in the ultrasonic probe 2 is identical with the axial direction of the artery 31. As pointed by the arrows A1, A2, A3 and so on, ultrasonic beams are sequentially transmitted at regular time intervals such that the beams scan the artery 31 in the axial direction in the scan region R1 of the ultrasonic probe 2. Also, the ultrasonic beams pointed by the arrows A1, A2, A3 and so on are reflected as ultrasonic echoes back toward the ultrasonic probe 2. As already described with reference to FIG. 2, each ultrasonic beam is produced by combining together a number of ultrasonic waves that have been transmitted from a plurality of ultrasonic vibrators 1 with their delays controlled.

In this case, if the arterial wall tissue of the artery 31 only dilated and contracted as the heart contracts, the measuring point M that has been set on the arterial wall tissue would always move parallel to the ultrasonic beams A1, A2, A3 and so on. In that case, the shape property or the qualitative property of the arterial wall tissue could be evaluated only with the ultrasonic beam that passes the measuring point. In other words, in the example shown in FIG. 4, the results of measurements done with the ultrasonic beam A2 would not affect the motion of the measuring point M perpendicular to the axial direction.

Actually, however, the arterial wall tissue produces an axial motion, too. That is why the ultrasonic diagnostic apparatus 20 shifts the measuring ultrasonic beam along the axis of the arterial wall tissue as the arterial wall tissue is displaced axially. This shift can be done by selecting one of the ultrasonic beams A1, A2, A3 and so on for scanning the scan region R1 according to the magnitude of displacement of the measuring point. More specifically, supposing a measuring point M that was located on the ultrasonic beam A1 at a time t=0 has moved to a location M′ in a predetermined amount of time t=t′ as a result of the axial motion of the arterial wall tissue, the ultrasonic beam A1 is selected at t=0 and the ultrasonic beam A3 is selected at t=t′ as an ultrasonic beam for evaluating the shape and attribute properties of the measuring point M that has been set on the arterial wall tissue.

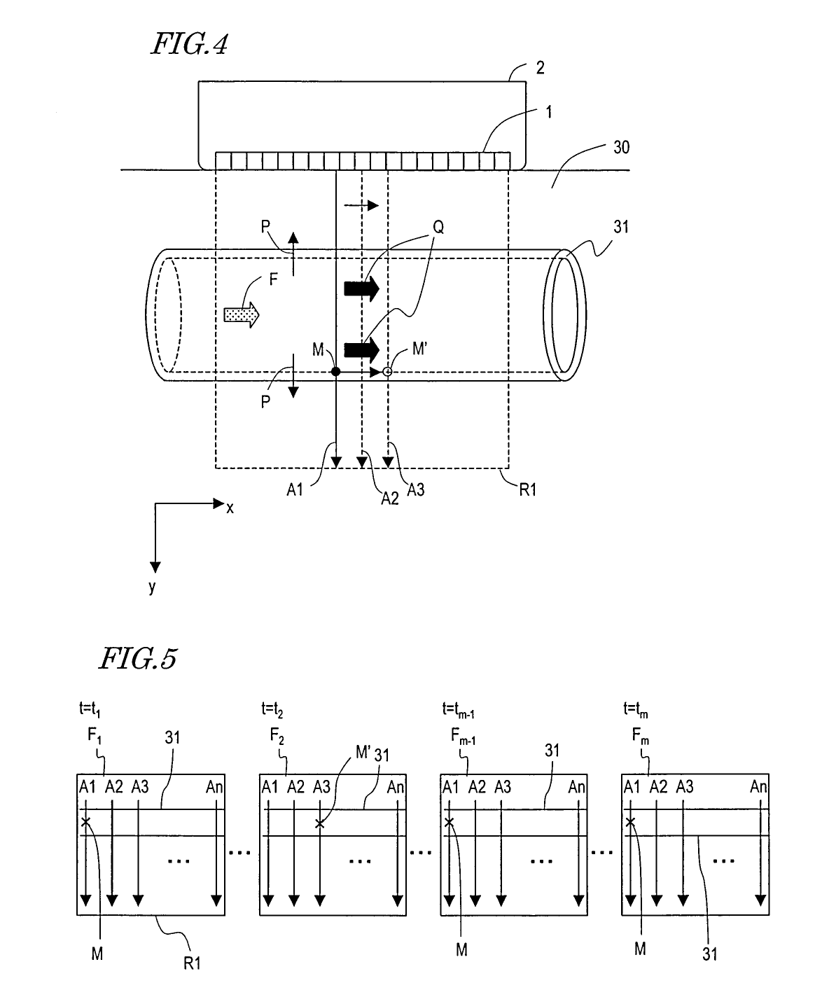

It depends on the axial velocity of the arterial wall tissue which ultrasonic beam should be selected every frame period. FIG. 5 schematically shows a relation between the ultrasonic beams that scan the scan region R1 and the measuring point M that has been set on the arterial wall tissue in axial motion in the ultrasonic diagnostic apparatus 20. If the shape or qualitative property of the scan region R1 is evaluated by scanning the scan region R1 with ultrasonic beams m times a cardiac cycle, frames F₁ through Fₘ are obtained from a time t=t₁ through a time t=tₘ. The ultrasonic beams A1 through An being transmitted to make a sequential scan in the respective frames remain at their respective locations.

As shown in FIG. 5, at the time t=t₁ when the frame F₁ is obtained, the measuring point M is located on the ultrasonic beam A1. Next, at the time t=t₂ when the frame F₂ is obtained, the measuring point M′ has shifted to a location on the ultrasonic beam A3 due to the axial motion of the arterial wall tissue. After that, the arterial wall tissue slowly gets back to its original location. And at times t=tₘ₋₁ and t=tₘ when the frame Fₘ₋₁ and Fₘ are obtained, the measuring point M is located on the ultrasonic beam A1 again. In this case, to evaluate the shape and qualitative property of the arterial wall tissue at the measuring point M, the ultrasonic beam A3 is selected in the frame F₂ and the ultrasonic beam A1 is selected in the other frames F₁, F_{M-1} and Fₘ.

Only the measuring point M is shown in FIG. 5. However, if the entire arterial wall tissue moves as the measuring point M is displaced axially, a shifted ultrasonic beam may be selected for not just the measuring point M but also any other measuring point. On the other hand, if the axial velocity changes from one location to another within the scan region R1 of the arterial wall tissue, one of the ultrasonic beams should be selected on a point-by-point basis. As described above, it also depends on the axial velocity of each measuring point on the arterial wall tissue which ultrasonic beam should be selected. If the property of the axial motion of the arterial wall tissue in one cardiac cycle is known in advance, then the signal processing section 13 may select an ultrasonic beam on a frame-by-frame basis according to the motion property and calculate the motion velocity of each measuring point, for example, using the ultrasonic beam selected.

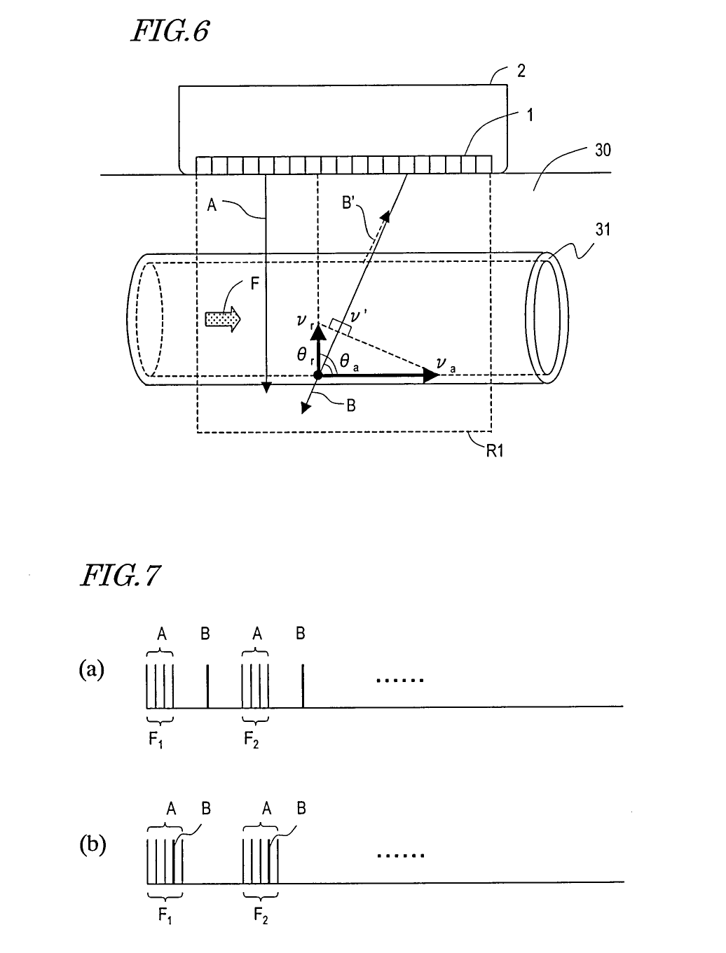

However, if the property of the axial motion of each measuring point on the arterial wall tissue is not known or if the axial motion of each measuring point needs to be calculated accurately, then the second ultrasonic beam described above is used. As shown in FIG. 6, the second ultrasonic beam B is transmitted from the ultrasonic probe 2 toward the artery 31 so as to define an angle of deviation θa with respect to the axis of the arterial wall tissue. The angle of deviation θa is different from that of the first ultrasonic beam A for inspecting the arterial wall tissue and should be within 90 degrees. The angle of deviation θa may be adjusted by controlling the time delays for the respective ultrasonic vibrators 1 included in the ultrasonic probe 2.

As shown in FIG. 6, a second ultrasonic echo B′, produced by getting the second ultrasonic beam B reflected from the posterior wall of the artery 31, is detected at the ultrasonic probe 2, and has its time delay controlled by the delay control section. After that, the receiving section 6 generates a second ultrasonic echo signal. The motion velocity detecting section 8 of the signal processing section 13 calculates the motion velocity v′ of each measuring point in the direction defined by the angle of deviation θa based on the second ultrasonic echo signal. The axial velocity va of each measuring point can be calculated as va=V′/cos θa. In this case, the motion velocity vᵣ of each measuring point perpendicular to the axial direction (i.e., in the radial direction) can be calculated as vr=v′ cos θᵣ, where θᵣ is the complementary angle of the angle of deviation θa. The arterial wall tissue is defined by two measuring points and the motion velocity of each measuring point defines the motion velocity of the arterial wall tissue.

In FIG. 6, only one second ultrasonic beam B is shown. However, just like the first ultrasonic beam A, a plurality of second ultrasonic beams B may be transmitted so as to scan the scan region R1. If the arterial wall tissue as a whole is moving axially at the same velocity within the scan region R1, the axial velocity just needs to be calculated with the only second ultrasonic beam B. On the other hand, if the axial velocity varies from one location to another on the arterial wall tissue, then a plurality of second ultrasonic beams B may be transmitted and the motion velocities may be calculated at multiple measuring points.

FIGS. 7(a) and 7(b) schematically show timings for calculating the axial motion velocities of the arterial wall tissue using the second ultrasonic beams B. If a frame Fₙ is obtained by scanning the tissue with the first ultrasonic beams A n times during one cardiac cycle as shown in FIG. 7, the second ultrasonic beam B may be transmitted between two frames as shown in FIG. 7(a) or while each frame is being scanned with the first ultrasonic beam A as shown in FIG. 7(b). Also, there is no need to transmit the second ultrasonic beam B for every frame but the number of times the second ultrasonic beams B are transmitted may be smaller than that of the frames. Furthermore, the motion velocities may be calculated by transmitting the second ultrasonic beams B only while the axial motion of the arterial wall tissue is significant during one cardiac cycle. It is at least preferable that the second ultrasonic beams B are transmitted synchronously with one cardiac cycle.

The computing section 9 of the signal processing section 13 receives the motion velocity vₐ thus calculated from the motion velocity detecting section 8 and selects one of the ultrasonic echo signals of the first group one frame after another based on the motion velocity va in order to calculate the shape property or the qualitative property at each measuring point. In this case, the ultrasonic echo signals of the first group may be either acquired in real time or have been stored in the received signal storage section 7. More specifically, the location to which each measuring point has been displaced at an arbitrary point in time may be figured out by sequentially integrating the motion velocities vₐ. Alternatively, the location to which each measuring point will be displaced at a frame a predetermined amount of time later may be calculated based on the motion velocity vₐ. As described above, if the motion velocity vₐ has not been calculated for each frame or found to be small as a result of measurement, then the first ultrasonic echo signal at the same location is selected continuously. By using the first ultrasonic echo signal that has been selected for each measuring point in this manner, either the magnitude of displacement or motion velocity and then the thickness variation are calculated.

The ultrasonic diagnostic apparatus 20 selects an ultrasonic echo signal for calculating the thickness variation or the elasticity between measuring points on a frame period basis according to the axial velocity of the arterial wall tissue. The motion velocity at each measuring point may be calculated by the same method as conventional ones using the ultrasonic beam selected. Consequently, the elasticity of an arterial wall tissue, which is having a two-dimensional motion on a cross section including the center axis of the artery, can be calculated accurately even without using a large scale computing circuit.

Hereinafter, as a specific exemplary measurement that uses the ultrasonic diagnostic apparatus of the present invention, an example in which the elasticity of an arterial wall tissue is calculated by a restricted minimum square method using the ultrasonic diagnostic apparatus 20 will be described.

First, it will be described how to make measurements in a situation where the arterial wall tissue is not moving axially. The arterial wall tissue having no axial motion will move only radially, i.e., perpendicularly to the axis of the artery. That is why the elasticity of each portion of the arterial wall can be calculated only on the ultrasonic echo signal generated from the ultrasonic beam that passes that location.

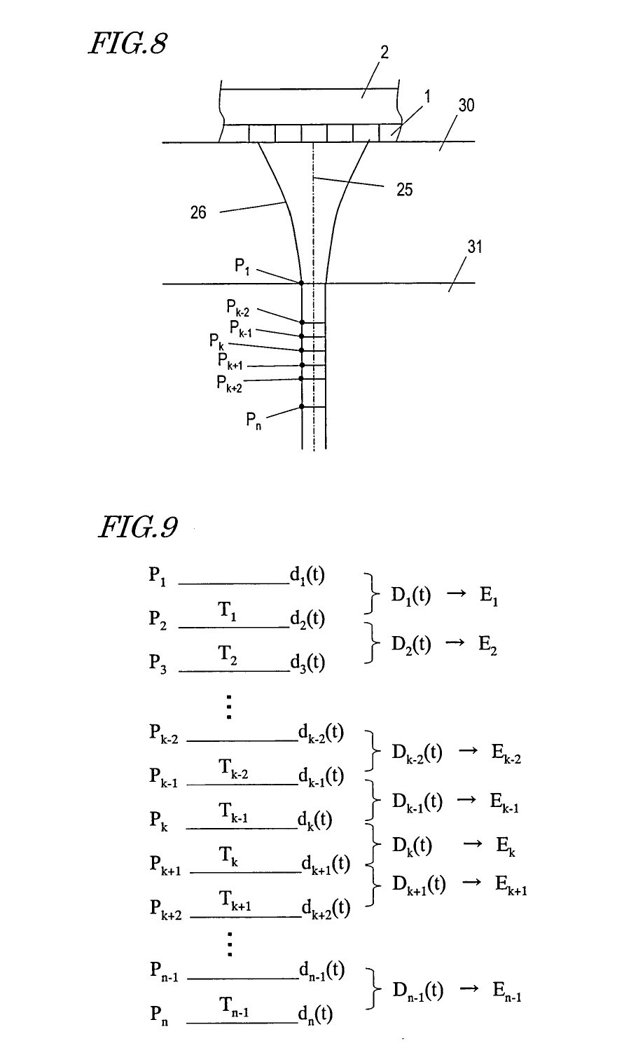

As shown in FIG. 8, the first ultrasonic beam 26, transmitted from the ultrasonic probe 2, propagates through the artery 31 of the living tissue 30. In the meantime, a portion of the ultrasonic wave is reflected by the arterial wall tissue of the artery 31 back toward the ultrasonic probe 2 and received there as a first ultrasonic echo. And a first ultrasonic echo signal is supplied to the signal processing section 13. The first ultrasonic echo signal is processed as a time series signal r(t). The closer to the ultrasonic probe 2 the tissue that has reflected the ultrasonic wave to produce the time series signal, the closer to the origin the signal is located on the time axis. The width (i.e., beam spot size) of the first ultrasonic beam 26 can be controlled by changing the time delay.

A plurality of measuring points Pₙ of the artery 31, which are located on an acoustic line 25 of the first ultrasonic beam 26, are arranged at regular intervals in the order of P₁, P₂, P₃, . . . , Pₖ, . . . and Pₙ (where n is natural number that is equal to or greater than three) where P₁ is a located closest to the ultrasonic probe 2. Supposing the coordinates that are defined in the depth direction with respect to the surface of the living tissue 30 as the origin are represented by Z₁, Z₂, Z₃, . . . , Zₖ, . . . and Zₙ, an ultrasonic wave reflected from a measuring point Pₖ is located at tₖ=2Zₖ/c on the time axis, where c is the velocity of the ultrasonic wave in the body tissue.

The reflected wave signal r(t) has its phase detected by the phase detecting section, provided for the motion velocity detecting section 8, and the phase-detected signal is split into a real part signal and an imaginary part signal, which are then passed through the filter section. Under the restriction that the amplitude does not change, but only the phase and reflection spot change, between the reflected wave signal r(t) and another reflected wave signal r(t+Δt) obtained after a very small amount of time Δt, the phase difference is calculated by a minimum square method so as to minimize the waveform mismatch between the reflected wave signals r(t) and r(t+Δt). The motion velocity Vₙ(t) of the measuring point Pₙ is derived from this phase difference and then integrated, thereby obtaining the magnitude of positional displacement dₙ(t).

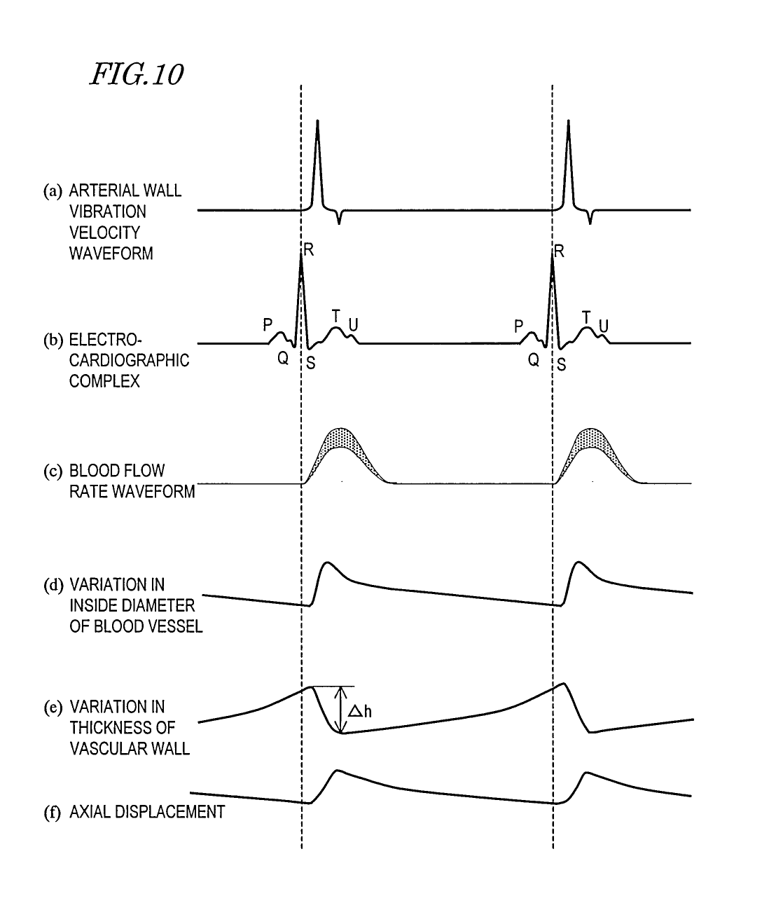

FIG. 9 shows the relationship between the measuring point Pₙ and the tissue under test Tₙ, of which the elasticity needs to be calculated. A tissue under test Tₖ is located between two adjacent measuring points Pₖ and Pₖ₊₁ so as to have a thickness h. That is to say, a number (n−1) of tissues under test T₁ through Tₙ₋₁ can be sampled from a number n of measuring points P₁ through Pₙ.

The variation Dₖ(t) in the thickness of the tissue under test Tₖ (i.e., the magnitude of its dilation, contraction or strain) is obtained as the difference between the magnitudes of positional displacement dₖ(t) and dₖ₊₁(t) of the measuring points Pₖ and Pₖ₊₁ (i.e., Dₖ(t)=dₖ₊₁(t)−dₖ(t)). If the tissue under test does not move axially, the difference in the magnitude of positional displacement between the measuring points always represents the thickness variation that is the magnitude of dilation, contraction or strain of the tissue under test.

The thickness of the tissue Tₖ of the arterial wall 31 varies when the blood flowing inside the arterial wall 31 changes with the cardiac rate. Thus, the elasticity Eₖ (i.e., the strain rate) of the tissue under test Tₖ in the vascular radial direction is given by:

Eₖ=(Δp×Hₖ)/Δhₖ

where Hₖ is the maximum thickness of the tissue under test Tₖ (i.e., the value associated with the lowest blood pressure), Δhₖ is the difference between the maximum and minimum variations Dₖ(t) in the thickness of the tissue under test, and Δp is pulse pressure that is the difference between the lowest and highest blood pressures.

In the example described above, the elasticity is calculated between two adjacent measuring points. However, the elasticity may also be calculated between two arbitrary ones of the multiple measuring points. In that case, the elasticity can be calculated in a similar manner by using the maximum thickness and the maximum and minimum thickness variations between the two points selected. For example, the thickness variation and elasticity between two points that have been set on the intima and the adventitia of the arterial wall may be calculated.

As described above, the tissue under test Tₖ moves axially. That is why the ultrasonic diagnostic apparatus of this preferred embodiment calculates the axial velocity of the tissue under test Tₖ using the second ultrasonic beam and selects one of the ultrasonic beams of the first group for use in the calculations according to the motion velocity. When the elasticity Eₖ is calculated, however, just the greatest thickness difference Δhₖ, which is the difference between the maximum and minimum thickness variations Dₖ(t) in one cardiac cycle, needs to be calculated and there is no need to calculate the magnitude of dilation or contraction of the tissue under test Tₖ continuously for one cardiac cycle.

Portions (a) through (e) of FIG. 10 show a waveform of the vibration velocity of an arterial wall in one cardiac cycle, an electrocardiographic complex, a waveform of a blood flow velocity, a waveform representing a variation in the inside diameter of a blood vessel, and a waveform representing a variation in the thickness of a vascular wall. As shown in portion (b) of FIG. 10, one ejection period of the heart normally begins with an R wave of the electrocardiographic complex and ends with a T wave thereof. When the R wave is produced, the heart starts to contract, too. At this point in time, no blood flow has been produced yet in the artery as shown in portion (c) of FIG. 10. That is why no shear stress is produced by the blood flow and no axial motion of the arterial wall occurs, either, as shown in portion (a) of FIG. 10. For these reasons, when or right after the R wave is produced, the blood vessel contracts most and the vascular wall thickens most in one cardiac cycle.

Soon after the R wave has been produced, the heart contracts to produce a blood flow. As a result, as shown in portions (d) and (e) of FIG. 10, the blood vessel dilates and the vascular wall decreases its thickness. Also, shear stress is produced by the blood flow and the arterial wall starts to move axially.

As shown in portion (b) of FIG. 10, a T wave of the electrocardiographic complex is produced at the end of a systolic period of the heart. At this point in time, the blood flow rate is the highest, the blood vessel dilates most and the vascular wall has the smallest thickness. As shown in portion (f) of FIG. 10, the axial displacement also has the greatest magnitude. Thereafter, the blood flow rate decreases gradually. Until another R wave of the electrocardiographic complex is produced, the inside diameter of the blood vessel decreases gradually, too, and the vascular wall gradually increases its thickness.

As can be seen from portion (e) of FIG. 10, the greatest thickness difference Δh of the vascular wall can be calculated by measuring the variations in the thickness of the vascular wall right after the R and T waves of the electrocardiographic complex. Therefore, to calculate the elasticity, just the thickness variations right after the R and T waves have been produced in one cardiac cycle need to be known. And to calculate the thickness variation, either motion velocities or locations may be figured out in sync with the R and T waves at two measuring points that define the thickness. More specifically, by transmitting the second ultrasonic beams when or right after the R and T waves are produced, the axial velocity between the two measuring points that define the thickness may be measured. And based on the results of measurements, one of the ultrasonic beams of the first group may be selected to find the results of measurements at the two measuring points.

For that purpose, an electrocardiograph may be connected as the biomedical signal detector 31 to the ultrasonic diagnostic apparatus 20 as shown in FIG. 1 and the second ultrasonic beams may be generated in response to the detection signals of the R and T waves on the electrocardiographic complex. And by calculating the axial velocities of the arterial wall tissue only at these times, the thickness variation of the arterial wall tissue can be measured accurately without increasing the computational complexity excessively.

In the preferred embodiment described above, the R and T waves of the electrocardiographic complex are detected by using an electrocardiograph as the biomedical signal detector 31. However, any other biomedical signal detector may also be used. For example, a phonocardiograph may also be used and the second ultrasonic beams may be transmitted synchronously with the generation of I-sound at the start of the ejection period of the heart and with the generation of II-sound when the aortic valve is closed after the heart has started to dilate.

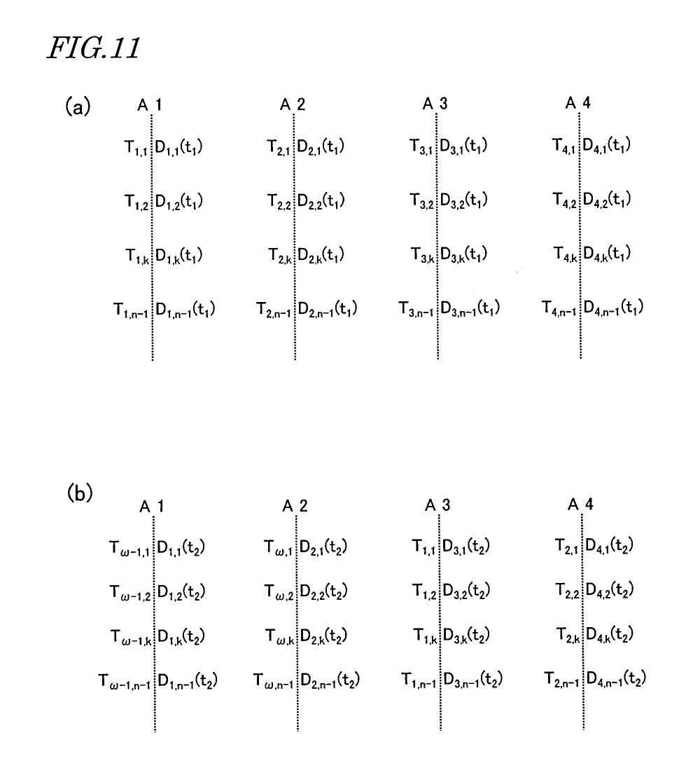

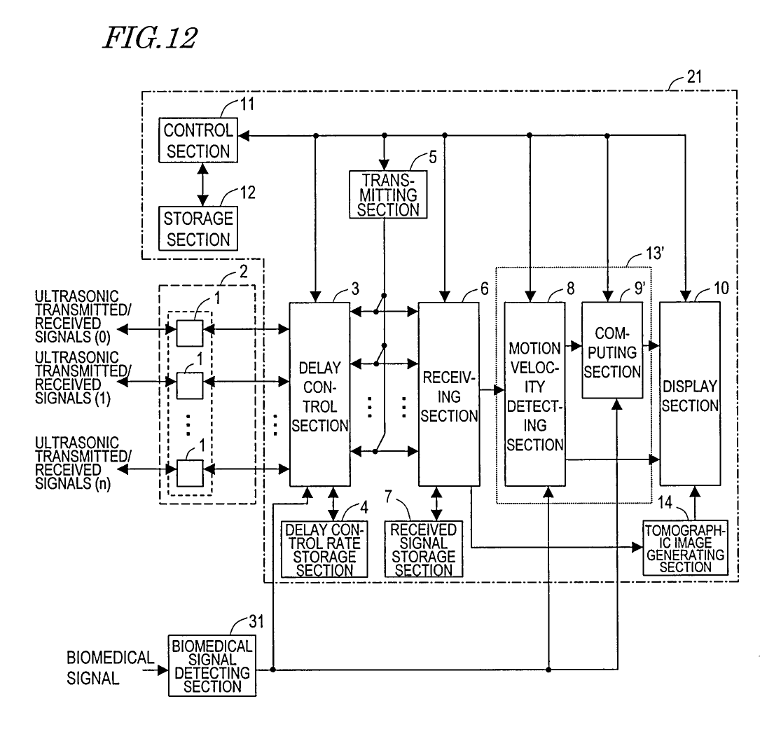

FIGS. 11(a) and 11(b) show the locations of the tissue under tests on the arterial wall at the time t=t₁ when the vascular wall has the greatest thickness and at the time t=t₂ when the vascular wall has the smallest thickness. As described above, these times are right after the R and T waves have been produced on the electrocardiographic complex. In these drawings, A1, A2, A3 and A4 denote the locations of first ultrasonic beams that are adjacent to each other and first ultrasonic echo signals obtained from their echoes. As shown in FIG. 11(a), tissue under tests T_{1,1}, through T_{1,n-1}, which are defined as tissues between the measuring points, are located on the first ultrasonic beams, and the thickness variations of these tissue under tests are identified by D_{1,1}(t₁) through D_{1,n-1}(t₁), respectively. In the same way, the tissue under tests on the first ultrasonic beams A2, A3 and A4 and their thickness variations are identified by T_{2,1} through T_{2,n-1} and D_{2,1}(t₁) through D_{2,1}(t₁), respectively.

As shown in FIG. 11(b), at the time t₂ when the vascular wall has the smallest thickness, the tissue under tests T_{1,1} through T_{1,n-1}, which were located on the first ultrasonic beam A1, are now located on the first ultrasonic beam A3 as a result of the axial motion of the artery. In the same way, the tissue under tests T_{2,1} through T_{2,n-1}, which were located on the first ultrasonic beam A2, are now located on the first ultrasonic beams A4 as a result of the axial motion of the artery. In this case, the thickness variations of the tissue under tests T_{1,1} through T_{1,n-1} and T_{2,1} through T_{2,n-1} are identified by D_{3,1}(t₂) through D_{3,n-1}(t₂) and D_{4,1}(t₂) through D_{4,n-1}(t₂), respectively. On the first ultrasonic beams A1 and A2, tissue under tests T_{ω-1,1} through T_{1,n-1}T and T_{ω,1} through T_{1,n-1}T, which were outside of the measuring range at the time t₁, are now located.

Consequently, supposing the time t₁ is a reference time, the greatest thickness differences Δh_{1,1} through Δh_{1,n-1} of the tissue under tests T_{1,1} through T_{1,n-1} on the first ultrasonic beam A1 can be calculated by D_{1,1}(t₁)−D_{3,1}(t₂) through D_{1,n-1}(t₁)−D_{3,n-1} (t₂), respectively. The elasticities can be calculated by the Equation (1) described above. The thickness variations on the respective first ultrasonic beams at the time t₂ when the vascular wall has the smallest thickness can be calculated by the same method as conventional ones based on the first ultrasonic echo signals generated by getting the respective ultrasonic beams reflected. As a result, the amount of computations to be done to figure out the elasticity is almost the same as a situation where the elasticity is figured out by a conventional method.

As described above, in calculating the elasticity of the arterial wall, one of the first ultrasonic echo signals obtained by scanning the tissue with the ultrasonic beams in a frame period including a time when the arterial wall has the greatest thickness and another one of the first ultrasonic echo signals obtained by scanning the tissue with the ultrasonic beams in a frame period including a time when the arterial wall has the smallest thickness may be selected according to the axial velocity or the magnitude of axial displacement. Also, at the time when the arterial wall has the greatest thickness, the axial motion of the arterial wall is the smallest and the magnitude of axial displacement is zero. That is why the second ultrasonic beam may be transmitted within or around a frame period including a time when the arterial wall has the smallest thickness, and the axial velocity or the magnitude of axial displacement of the arterial wall may be calculated based on the resultant second ultrasonic echo signal. The elasticity changes periodically in sync with one cardiac cycle. For that reason, such a selection of the first ultrasonic echo signal may be made every cardiac cycle.

### Embodiment 2

Hereinafter, a second preferred embodiment of an ultrasonic diagnostic apparatus according to the present invention will be described with reference to the accompanying drawings. The ultrasonic diagnostic apparatus 21 of this preferred embodiment detects either the axial velocity or the magnitude of axial displacement of an artery. If the artery is found to be moving axially, the apparatus tells the operator the fact that measurements cannot be done properly due to the axial motion of the artery. FIG. 12 is a block diagram of the ultrasonic diagnostic apparatus 21 of this preferred embodiment, which includes the delay control section 3, the delay control rate storage section 4, the transmitting section 5, the receiving section 6, the received signal storage section 7, a signal processing section 13′, the display section 10, the control section 11, the storage section 12, and the tomographic image generating section 14.

As in the first preferred embodiment described above, the transmitting section 5 drives the respective ultrasonic vibrators 1 of the ultrasonic probe 2, thereby generating an ultrasonic transmission signal to transmit first and second ultrasonic beams to the arterial wall tissue. The ultrasonic transmission signal thus generated is input to the delay control section 3, where the delays are controlled such that the respective ultrasonic vibrators 1 are driven at predetermined timings.

The ultrasonic echoes, produced by getting the first and second ultrasonic beams reflected from the arterial wall, are received at the respective ultrasonic vibrators 1 of the ultrasonic probe 2, have their delays controlled by the delay control section 3, and then are synthesized and amplified by the receiving section 6, which outputs first and second ultrasonic echo signals.

The signal processing section 13′ includes a motion velocity detecting section 8 and a computing section 9′. The motion velocity detecting section 8 detects the motion velocity of the arterial wall tissue at each measuring point, or the magnitude of displacement that is an integrated value thereof, based on the first ultrasonic echo signal. Also, the motion velocity detecting section 8 detects the axial velocity of the arterial wall tissue at each measuring point, or the magnitude of axial displacement thereof, based on the second ultrasonic echo signal.

In estimating the thickness variation or the elasticity using this ultrasonic diagnostic apparatus 21, it is important to accurately measure the maximum and minimum thicknesses of the arterial wall tissue in one cardiac cycle. As already described for the first preferred embodiment, when the arterial wall tissue has the smallest thickness, the magnitude of axial displacement of the arterial wall is maximized. That is why either within or around a frame period including a time when the arterial wall tissue has the smallest thickness, the second ultrasonic beam is preferably transmitted and the axial velocity or the magnitude of axial displacement at each measuring point on the arterial wall tissue is preferably calculated based on the resultant second ultrasonic echo signal.

Based on the motion velocities, or the magnitudes of displacement, at respective measuring points of the arterial wall tissue, which have been derived from the first ultrasonic echo signals, the computing section 9 calculates either the thickness variation, or the elasticity, between the measuring points on the arterial wall tissue. Also, the computing section 9 compares the axial velocity or magnitude of axial displacement at each measuring point on the arterial wall tissue, on which the thickness variation or the elasticity has been calculated, to a predetermined threshold value. If the motion velocity or the magnitude of displacement is greater than the threshold value, the computing section 9 outputs neither the thickness variation nor the elasticity of that arterial wall tissue to the display section 10. Alternatively, the computing section 9 may replace the thickness variation or elasticity value thus obtained with a value indicating that the thickness variation or elasticity value is unusual (e.g., a predetermined negative value). On the other hand, if the motion velocity or the magnitude of displacement is equal to or smaller than the threshold value, the computing section 9 outputs the thickness variation or the elasticity of that portion of the arterial wall tissue to the display section 10.

The tomographic image generating section 14 generates a tomographic image based on the first ultrasonic echo signal supplied from the receiving section 9. For example, the tomographic image generating section 14 transforms the amplitude intensity of the first ultrasonic echo signal into the luminance information of the image to be presented on the display section, thereby generating a B-mode tomographic image.

The display section 10 presents the tomographic image supplied from the tomographic image generating section 14 and the thickness variation or elasticity of each arterial wall tissue supplied from the computing section 9′ such that these two types of images are superimposed one upon the other on the screen.

Hereinafter, the procedure of measurements carried out by the ultrasonic diagnostic apparatus 21 will be described with reference to the flowchart shown in FIG. 13.

First, in Step 112, the transmitting section 5 transmits an ultrasonic wave from the ultrasonic probe 2 toward an organism including an arterial blood vessel. The ultrasonic echoes, generated by getting the transmitted ultrasonic wave reflected by the organism, are received through the ultrasonic probe 2 at the receiving section 6. The ultrasonic wave transmitted includes first and second ultrasonic beams. And the receiving section 6 outputs first and second ultrasonic echo signals based on these reflected echoes.

Next, in Step 113, the motion velocity detecting section 8 of the signal processing section 13 detects either the motion velocity or the magnitude of displacement at each measuring point on the arterial wall tissue based on the first ultrasonic echo signal.

Subsequently, in Step 114, the motion velocity detecting section 8 detects the axial velocity at each measuring point based on the second ultrasonic echo signal and may also calculate the magnitude of displacement.

Thereafter, in Step 115, based on the motion velocity or magnitude of displacement at each measuring point, the computing section 9′ calculates the thickness variation or elasticity between the measuring points on the arterial wall tissue.

Next, in Step 116, the motion velocity or magnitude of displacement at each arterial wall tissue, for which the thickness variation or the elasticity has been calculated, is compared to a threshold value. If the thickness variation or the elasticity is greater than the threshold value, the computing section 9′ stops outputting the thickness variation nor the elasticity of that arterial wall tissue to the display section 10 such that neither the thickness variation nor the elasticity is presented on the display section 10. Instead, the computing section 9′ outputs only the thickness variation or the elasticity of that arterial wall tissue, which is smaller than the threshold value, to the display section 10. On the other hand, if the motion velocities or the magnitudes of displacement of all arterial wall tissues are equal to or smaller than the threshold value, the computing section 9′ outputs the thickness variations or the elasticities calculated for the entire arterial wall tissue to the display section 10.

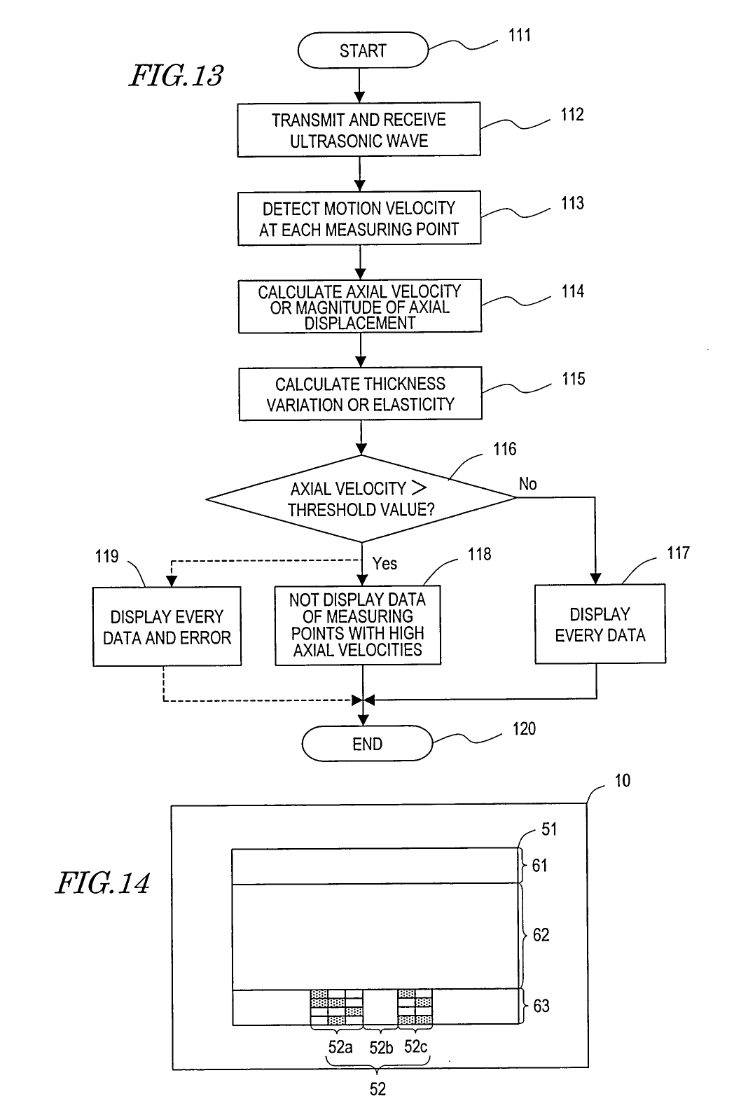

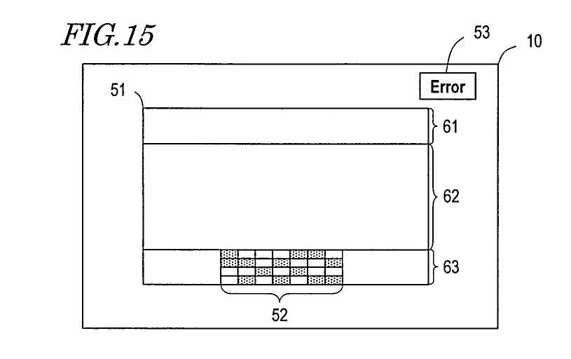

FIG. 14 schematically shows exemplary images presented on the display section 10 of the ultrasonic diagnostic apparatus 21. As shown in FIG. 14, a tomographic image 51 of the measuring region is presented on the display section 10. The tomographic image 51 shows an artery anterior wall 61, a blood vessel lumen 62, and the artery posterior wall 63. As the measuring region is now set on the artery posterior wall 63, a two-dimensional map image 52 representing the elasticity or thickness variation is superimposed on the artery posterior wall 63 of the tomographic image 51.

On the two-dimensional map image 52, the thickness variation or elasticity of the arterial wall tissue is presented in gray scales or color tones corresponding to its values on areas 52a and 52c. On the other area 52b, no thickness variation or elasticity of the arterial wall tissue but an image of the artery posterior wall 63 is presented. Thus, the operator can see easily that the arterial wall tissue is moving axially in the area 52b and the elasticity could not be calculated accurately.

As described above, according to this preferred embodiment, the portion in which the axial motion of the arterial wall prevents the thickness variation or the elasticity from being calculated accurately is detected and the thickness variation or elasticity of only the portion where measurements could be done accurately is presented on the display section. Consequently, the operator can make a proper diagnosis based on the information presented on the display section.

In the preferred embodiment described above, the computing section 9′ detects a portion in which the axial motion of the arterial wall prevents the thickness variation or the elasticity from being calculated accurately. However, if a portion of the arterial wall tissue within the measuring region is moving axially, either character information or image information telling the operator that fact may be presented on the display section 10 and the elasticity may be displayed as it is. More specifically, in Steps 116 and 119 shown in FIGS. 13 and 14, the computing section 9′ compares either the motion velocity or the magnitude of displacement of each arterial wall tissue, for which the thickness variation or elasticity has been calculated, to a threshold value. If any tissue has a thickness variation or elasticity that is greater than the threshold value, then the computing section 9′ generates a piece of information 53 indicating that the measurements could not be done properly as shown in FIG. 14, outputs a signal to the display section 10 and gets every thickness variation or elasticity calculated presented on the display section 10. Even by making such a presentation, the operator can also see easily that the measurements could not be done properly. In addition to displaying the piece of information 53 telling that the measurements could not be done properly, the portion in which the thickness variation or elasticity cannot be calculated accurately may be detected and the elasticity of that portion may not be presented as shown in FIG. 13.

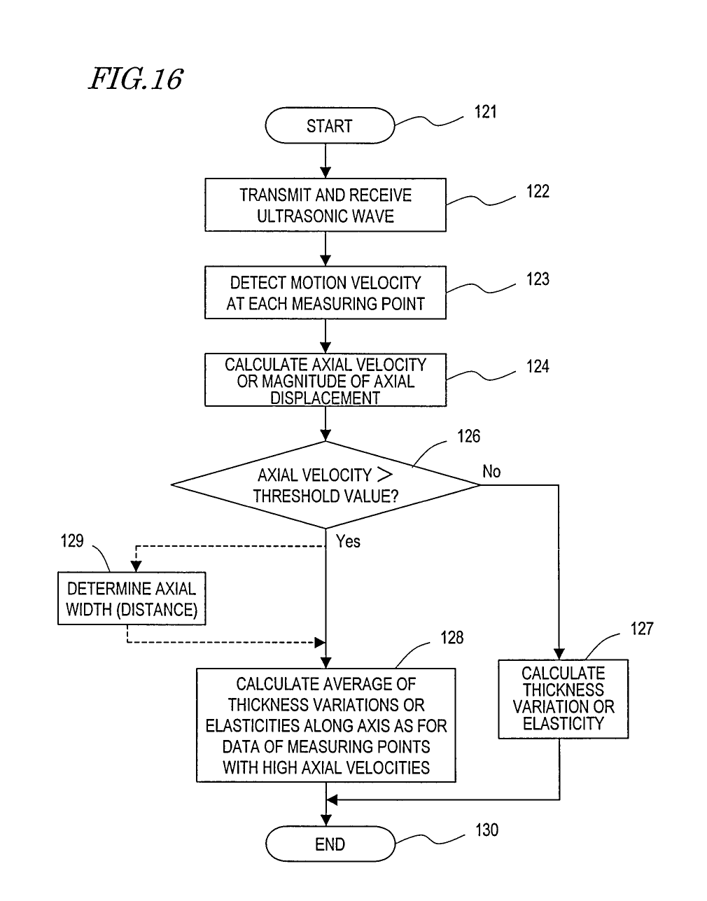

Also, if the arterial wall is moving axially, the average of the thickness variations or elasticities of multiple arterial wall tissues may be calculated along the axis of the arterial wall tissues and the averaged elasticity may be presented on the display section 10. Hereinafter, such a preferred embodiment will be described with reference to the flowchart of FIG. 16.

First, in Steps 112, 113 and 114, first and second ultrasonic beams are transmitted, the motion velocity or magnitude of displacement at each measuring point on the arterial wall tissue is calculated based on first and second ultrasonic echo signals received, and the axial velocity or magnitude of axial displacement at each measuring point is also calculated based on the second ultrasonic echo signal.

Next, in Step 116, the motion velocity or magnitude of displacement at each measuring point is compared to a threshold value. Subsequently, in Step 118, if there is any measuring point where the motion velocity or magnitude of displacement is greater than the threshold value and if a tissue is interposed between multiple measuring points where the axial velocities are greater than the threshold value, the thickness variations or elasticities are averaged in the axial direction. More specifically, first, the thickness variations or elasticities of tissues that are interposed between respective measuring points are calculated by the same method as a conventional one. Next, as for a tissue interposed between multiple measuring points at which the axial velocities are greater than the threshold value, either the thickness variations or elasticities that have been calculated for a predetermined number of (e.g., two) axially adjacent tissues are averaged.

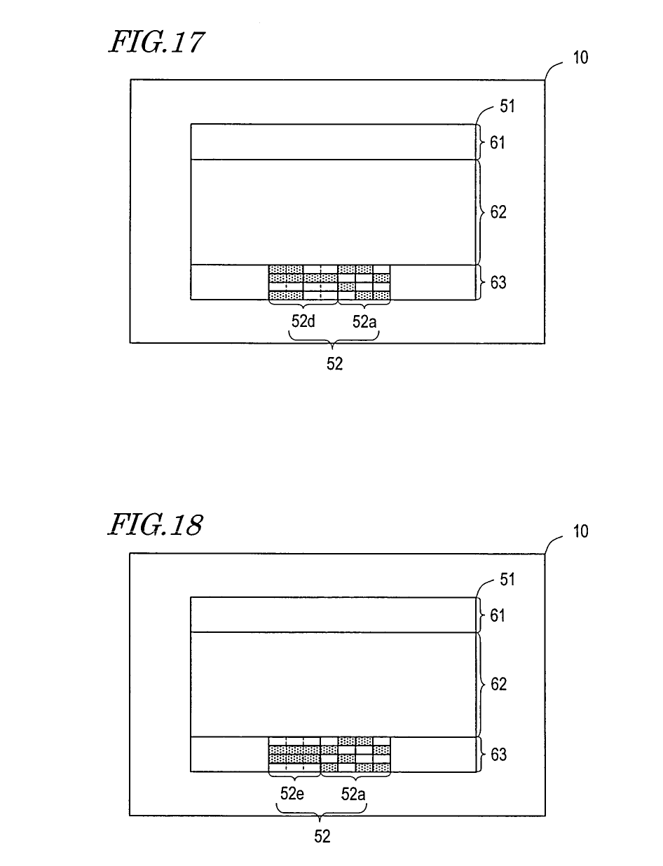

FIG. 17 schematically shows exemplary images on the display section 10 on which the elasticity that has been calculated in this manner is presented. As shown in FIG. 17, a tomographic image 51 of the measuring region is presented on the display section 10. The tomographic image 51 shows an artery anterior wall 61, a blood vessel lumen 62, and the artery posterior wall 63. As the measuring region is now set on the artery posterior wall 63, a two-dimensional map image 52 representing the elasticity or thickness variation is superimposed on the artery posterior wall 63 of the tomographic image 51.

On the two-dimensional map image 52, the thickness variation or elasticity of the arterial wall tissue is presented in gray scales or color tones corresponding to its values on areas 52a and 52c. On the other area 52b, the elasticities of axially adjacent tissues have been averaged and the average elasticity is shown as if the adjacent tissues were a single continuous tissue. Thus, the computational error of the elasticity due to the axial motion of the arterial wall tissue can be minimized.

The number of tissues for which the average needs to be calculated may be either determined in advance as described above or changed according to the motion velocity or magnitude of displacement of the arterial wall. In the latter case, in Step 116 shown in FIG. 16, for example, the motion velocity or magnitude of displacement at each measuring point is compared to a threshold value. Next, in Step 119, if there is any measuring point where the motion velocity or magnitude of displacement is greater than the threshold value, then the distance over which the average needs to be calculated in the axial direction is determined by the motion velocity or magnitude of displacement at each measuring point. Subsequently, in Step 118, the thickness variations or elasticities of all tissues interposed between the respective measuring points are calculated. Thereafter, as for a tissue interposed between multiple measuring points where the axial velocities are greater than the threshold value, the thickness variations or elasticities of the number of tissues that should be included in the distance determined in Step 119 are averaged.

FIG. 18 schematically shows exemplary images on the display section 10 on which the elasticity that has been calculated in this manner is presented. As shown in FIG. 18, the thickness variations or elasticities of a number of (e.g., three in this example) tissues to be included in the distance that has been determined by the motion velocity or the magnitude of displacement have been averaged on the area 52e of the two-dimensional map image 52. Since the number of tissues for which the average needs to be calculated is determined by the axial velocity or magnitude of axial displacement of the arterial wall, the computational error of the elasticity due to the axial motion of the arterial wall tissue can be further reduced.

As described above, according to this preferred embodiment, either the axial velocity or magnitude of axial displacement of the arterial wall is calculated using the second ultrasonic beam and the modes of presenting the thickness variation or elasticity are changed according to the motion velocity or the magnitude of positional displacement. That is why the operator can see appropriately that the axial motion of the arterial wall prevented the thickness variation or elasticity from being calculated accurately and can make a more accurate diagnosis using the ultrasonic diagnostic apparatus. Besides, since the thickness variation or the elasticity is calculated without taking the axial motion into consideration, the computational complexity does not increase and no high-performance computer is needed, either. Consequently, an ultrasonic diagnostic apparatus can be provided at a reduced cost.

### INDUSTRIAL APPLICABILITY

The present invention can be used effectively in an ultrasonic diagnostic apparatus for evaluating the shape property or qualitative property of a living tissue. Among other things, the present invention can be used particularly effectively in an ultrasonic diagnostic apparatus for diagnosing atherosclerosis by measuring the elasticity of the artery.

1. An ultrasonic diagnostic apparatus for evaluating a shape property or a qualitative property of an arterial wall tissue of an organism, the apparatus comprising:
a delay control section for controlling delays for respective ultrasonic vibrators included in an ultrasonic probe;
a transmitting section for driving the ultrasonic probe under the control of the delay control section such that the ultrasonic probe transmits a first ultrasonic beam toward multiple different locations within a scan region, which is defined along the axis of the organism's artery, every predetermined frame period;
a receiving section for receiving a plurality of ultrasonic echoes, generated by getting the first ultrasonic beam reflected by the arterial wall, at the ultrasonic probe every predetermined frame period, thereby outputting a first group of ultrasonic echo signals; and
a signal processing section for calculating a thickness variation, or the elasticity, of the arterial wall tissue between multiple measuring points that have been set on the arterial wall tissue in response to the first group of ultrasonic echo signals,
wherein the signal processing section selects one of the ultrasonic echo signals of the first group every frame period according to an axial velocity of the arterial wall tissue to make calculations at each said measuring point.

2. The ultrasonic diagnostic apparatus of claim 1, wherein the signal processing section includes a motion velocity detecting section, and
wherein the transmitting section transmits a second ultrasonic beam, and
wherein the receiving section outputs a second group of ultrasonic echo signals, which are generated by getting the second ultrasonic beam reflected by the arterial wall, and
wherein the motion velocity detecting section calculates the axial velocity of the arterial wall tissue based on the second group of ultrasonic echo signals.

3. The ultrasonic diagnostic apparatus of claim 2, wherein the first and second ultrasonic beams have mutually different angles of deviation.

4. The ultrasonic diagnostic apparatus of claim 3, wherein the delay control section changes, at regular intervals, delay control rates to transmit the second ultrasonic beam.

5. The ultrasonic diagnostic apparatus of claim 3, wherein the delay control section receives a biomedical signal, containing information about the organism, and changes delay control rates to transmit the second ultrasonic beam at an interval that agrees with one cycle of the biomedical signal.

6. The ultrasonic diagnostic apparatus of claim 5, wherein the cycle of the biomedical signal is a cardiac cycle.

7. The ultrasonic diagnostic apparatus of claim 2, wherein the first ultrasonic beam is substantially perpendicular to the axis of the artery and the second ultrasonic beam is not perpendicular to the axis of the artery.

8. The ultrasonic diagnostic apparatus of claim 1, wherein the measuring points are arranged two-dimensionally, and wherein the computing section calculates the thickness variation, or the elasticity, of the arterial wall tissue two-dimensionally.

9. The ultrasonic diagnostic apparatus of claim 8, further comprising a display section for presenting results of calculations done by the computing section as a two-dimensional map.

10. A method for getting an ultrasonic diagnostic apparatus controlled by a control section of the apparatus, the method comprising the steps of:
transmitting a first ultrasonic beam from an ultrasonic probe toward multiple different locations within a scan region, which is defined along the axis of an organism's artery, every predetermined frame period;
receiving a plurality of ultrasonic echoes, generated by getting the first ultrasonic beam reflected by the arterial wall of the artery, at the ultrasonic probe every predetermined frame period, thereby generating a first group of ultrasonic echo signals;
selecting one of the ultrasonic echo signals of the first group every frame period according to an axial velocity of the arterial wall tissue to make calculations at each measuring point; and
calculating a thickness variation, or the elasticity, of the arterial wall tissue between at least two of multiple measuring points that have been set on the arterial wall tissue in response to the ultrasonic echo signal selected from the first group.

11. The method of claim 10, wherein the step of calculating includes the steps of:
transmitting a second ultrasonic beam toward the artery to obtain a second group of ultrasonic echo signals, which are generated by getting the second ultrasonic beam reflected by the arterial wall, and
calculating the axial velocity of the arterial wall tissue based on the second group of ultrasonic echo signals.

12. The method of claim 11, comprising the step of transmitting the second ultrasonic beam at an interval that agrees with one cycle of a biomedical signal containing information about the organism.

13. The method of claim 12, wherein the cycle of the biomedical signal is a cardiac cycle.
